(19) United States
(12) Patent Application Publication
(10) Pub. No.: US 2025/0244154 A1
(43) Pub. Date: Jul. 31, 2025

(54) LIQUID RATE TEST FROM SAMPLING COLLECTION POINTS

(71) Applicant: SAUDI ARABIAN OIL COMPANY, Dhahran (SA)

(72) Inventors: Rayed Mohammed Al-Zayer, Tarout (SA); James Ohioma Arukhe, Dhahran (SA); Suhail Abdullah Samman, Riyadh (SA)

(73) Assignee: SAUDI ARABIAN OIL COMPANY, Dhahran (SA)

(21) Appl. No.: 18/424,475

(22) Filed: Jan. 26, 2024

(51) Int. Cl. G01F 9/00; C02F 1/00; C02F 103/10; E21B 43/34; E21B 49/08; G01N 33/28

(52) U.S. Cl. CPC G01F 9/005; C02F 1/008; E21B 43/34; E21B 49/086; G01N 33/2847; C02F 2103/10; C02F 2209/40

(57) ABSTRACT

Systems and methods for measuring a fluid flow rate from a production well including a flow line for transporting produced fluids from the production well to a gas-oil separation plant, and a sample system. The sample system includes a sample vessel connected to a sample point port on the flow line. The sample vessel includes an inlet port, a sight glass having a calibrated volumetric scale, an outlet port, and a valve system for redirecting fluid flow from the flow line to the sample vessel via the sample point port. Methods include producing fluid from the production well and transporting produced fluid from the production well to the gas-oil separation plant, measuring a flow rate of produced fluid from the well and quantifying a water content of the produced fluid, and adjusting an operating condition of the gas-oil separation plant based upon the measured flow rate and water content.

### BACKGROUND

Devices for measuring individual flow rates of fluids in a produced fluids mixture are commonly used in oilfield operations. Knowledge of the individual fluid flow rates allows for reservoir management, production allocation, operational control, and field development. Accurate flow rate measurement of produced fluids such as oil and gas are therefore important for maximizing production and minimizing cost in oil and gas operations.

Current measurement techniques may require frequent calibrations and maintenance, large operation space, costly monitoring for corrosion and safety, and specialized equipment. In addition, current methods used to measure flow rates of produced fluids may be inaccurate in high water cut or high gas:oil ratio wells.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a system for measuring a fluid flow rate from a well, including a water flow rate. The system includes a production well, a flow line for transporting produced fluids from the production well to a gas-oil separation plant, and a sample system. The sample system includes a sample vessel fluidly connected to a sample point port disposed on the flow line. The sample vessel includes an inlet port fluidly connected to the sample point port via an inlet valve, disposed on an inlet flow line, a temperature gauge configured to measure a temperature of fluid received from the sample point port, a first pressure gauge configured to measure a pressure of fluid received from the sample point port, a second pressure gauge configured to measure a total vessel pressure, fluidly connected to a pressure release line, where the pressure release line is configured to vent a gas from the sample vessel. The sample vessel also includes an outlet port fluidly connected to a fluid discharge line via an outlet valve, a sight glass having a calibrated volumetric scale, where the sight glass is integrally formed in a vessel body and a valve system for redirecting fluid flow from the flow line to the sample vessel via the sample point port.

In another aspect, embodiments disclosed herein relate to a method for controlling gas-oil separation plant operations using the system including producing fluid from the production well and transporting produced fluid from the production well to the gas-oil separation plant, measuring a flow rate of produced fluid from the well and quantifying a water content of the produced fluid, and adjusting an operating condition of the gas-oil separation plant based upon the measured flow rate and water content.

Other aspects and advantages of the claimed subject matter will be apparent from the following description and the appended claims.

### BRIEF DESCRIPTION OF DRAWINGS

FIGS. 1A-1C show systems in accordance with one or more embodiments.

FIG. 2 shows a sampling system in accordance with one or more embodiments.

FIG. 3 is a flowchart of a method in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (for example, first, second, third) may be used as an adjective for an element (that is, any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

It is to be understood that the singular forms “a,” “an,” and “the” include plural referents unless the context clearly dictates otherwise. Thus, for example, reference to “a fluid sample” includes reference to one or more of such samples.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowcharts may be omitted, repeated, and/or performed in a different order than the order shown. Accordingly, the scope of the invention should not be considered limited to the specific arrangement of steps shown in the flowcharts.

Although multiply dependent claims are not introduced, it would be apparent to one of ordinary skill that the subject matter of the dependent claims of one or more embodiments may be combined with other dependent claims.

Embodiments disclosed herein generally relate to systems and methods for measuring a fluid flow rate from a well and controlling gas-oil separation plant operations based on the measured fluid flow rate. The technique of one or more embodiments includes a small, fabricated apparatus to measure the oil, water rates, and water cut for the oil wells.

System for Measuring a Fluid Flow Rate from a Well

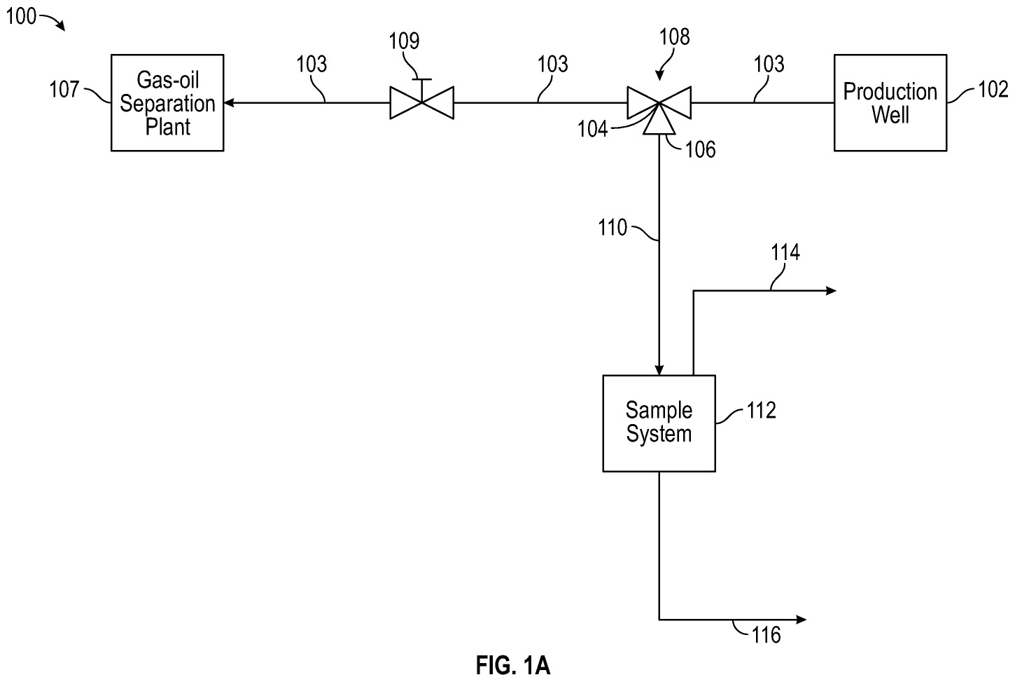

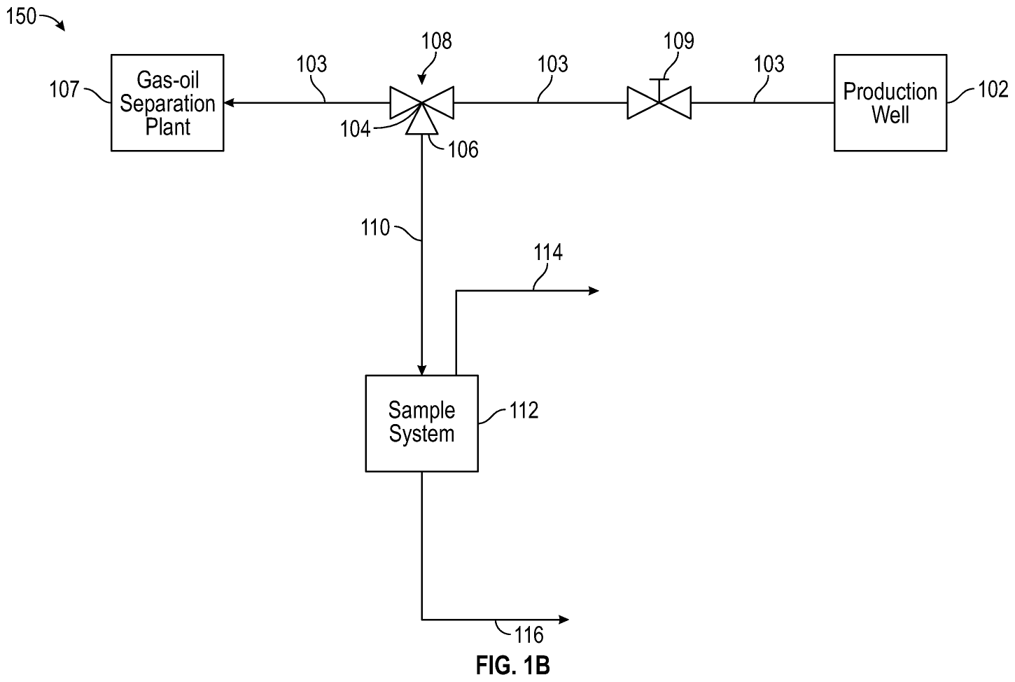

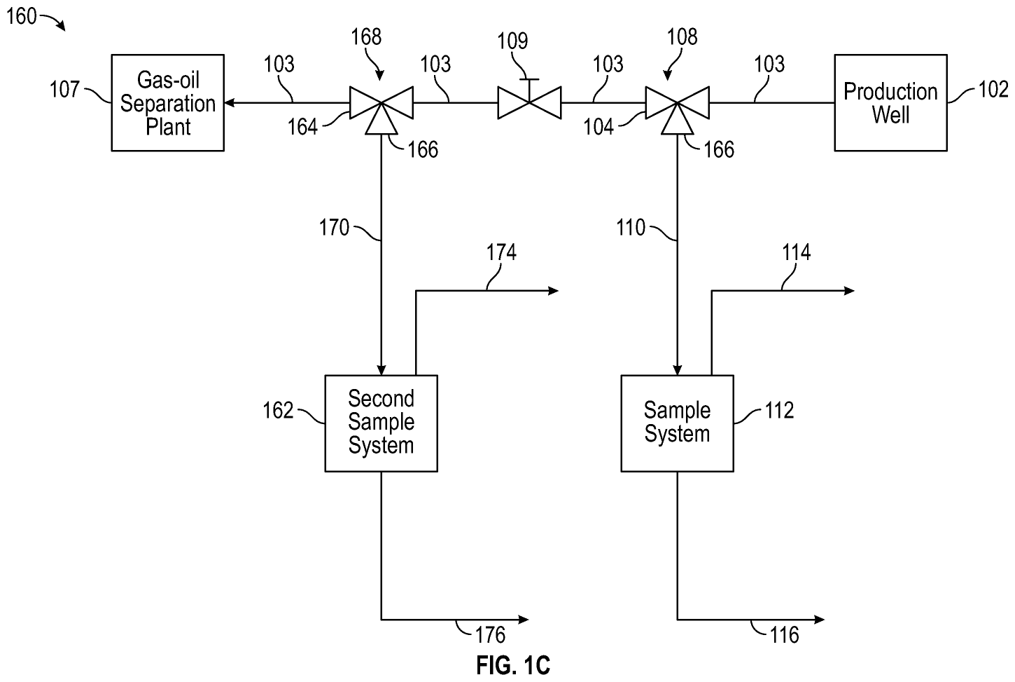

One or more embodiments relate to a system for measuring a fluid flow rate from a well, including a water flow rate. FIG. 1A illustrates a system 100 according to one or more embodiments. The system 100 includes a production well 102, a flow line 103, a sample system 112, and a valve system 108.

The production well 102 in the system 100 of FIG. 1A of one or more embodiments is defined as any well used to retrieve oil or gas from an underground reservoir.

In one or more embodiments, the production well 102 is fluidly connected to a gas-oil separation plant 107 by a flow line 103. The flow line 103 transports produced fluid from the production well 102 to the gas-oil separation plant 107.

Produced fluids transported by the flow line 103 according to one or more embodiments may be any fluids produced by a production well. For example, produced fluids may include water, natural gas, oil, dissolved solids, and the like.

The gas-oil separation plant 107 of one or more embodiments may be any gas-oil separation plant known in the art capable of separating natural gas from liquids, including oil and water. The gas-oil separation plant of one or more embodiments may be a temporary plant or a permanent plant. The gas-oil separation plant may include a two-phase separator configured to separate gas from oil, a three-phase separator configured to separate gas, oil, and water, and combinations thereof. The separator may be horizontal, vertical, or spherical. One or more separators may be used in the gas-oil separation plant. In general, a gas-oil separation plant may include a test separator vessel which may be connected to the wellhead after the choke valve. The test separator vessel separates produced fluids based on their density. For example, solids, such as sand, settle to the bottom of the separator. Denser liquids such as oil and water are drawn from a valve at the bottom of the separator and less dense fluids, such as gas, migrate to the top of the separator vessel where they may be withdrawn. The withdrawn gas may be flared or captured for further use.

Keeping with FIG. 1A, in one or more embodiments, a valve system 108 is disposed on the flow line 103. The valve system 108 may redirect fluid flow from the flow line 103 to a sample system 112 via a sample point port 104.

The valve system 108 of one or more embodiments includes any valve or valves capable of diverting flow of fluids, such as produced fluids, from one direction of flow to another direction of flow. The valve system may be a manually operated, actuatively operated, or automatically operated. Valves within the valve system may include one or more of a ball valve, a butterfly valve, a check valve, a gate valve, a globe valve, and the like. The valve system may include one or more different types of valves. As will be understood by one of ordinary skill in the art, the types and size of valves included in the valve system may vary depending on the specifics of the surrounding system. The valve system may include three-way valves or may include multiple valves configured appropriately to direct flow in one direction or another. The valve system may also include appropriate purge lines to allow for purging of any solids accumulation before the sample flow is directed toward the sample vessel.

In the system 100 of FIG. 1A, a choke valve 109 may be included at a location on the flow line 103 which is downstream of the valve system 108 leading to the sample point port 104 and upstream of the gas-oil separation plant 107. In one or more embodiments, the sample point port 104 is advantageously upstream of the choke valve 109 to ensure turbulent flow of produced fluids into the valve system 108 which may lead to better mixing of water and oil in the produced fluids and subsequently a more accurate basic sediment and water (BS&W) measurement. In one or more embodiments, turbulent flow from the production well is advantageous because it may prevent premature settling of solids or separation of gases and liquids, resulting in “slug” flow, which may lead to inconsistent representative compositions of the fluid sample.

The choke valve 109 of one or more embodiments includes any choke valve known in the art. A choke valve allows an operator to adjust a pressure and flow according to the requirements of a system. The choke valve 109 may be manually operated or automatically operated by a system operator. Examples of choke valves include a needle and seat choke, a plug and cage choke, a multiple orifice valve, and the like.

In one or more embodiments, the valve system 108 is operated to allow fluid to flow from the flow line 103 through the sample point port 104 via an inlet valve 106. When the inlet valve 106 is opened, fluid flows through an inlet fluid line 110 to the sample system 112. A pressure release line 114 configured to vent a gas to atmosphere, a flare, or a gas collection system is fluidly connected to the sample system 112. A fluid discharge line 116 configured to discharge fluid is also fluidly connected to the sample system 112.

FIG. 1B shows another system 150 according to one or more embodiments. The components and their functions are identical to the description of FIG. 1A, above. In the system 150 of FIG. 1B, however, a choke valve 109 is located on the flow line 103 at a position upstream of the sample point port 104 and downstream of the production well 102.

FIG. 1C shows another system 160 according to one or more embodiments. The components and their functions are identical to the description of FIG. 1A, above. The system 160 of FIG. 1C, however, includes a plurality of sample systems, including a first sample system 112 and the components of sample system 112 discussed in FIG. 1A. The system 160 also includes a second sample system 162. The second sample system includes a second valve system 168 disposed on the flow line 103. The second valve system 168 may redirect fluid flow from the flow line 103 to the second sample system 162 via a second sample point port 164. The second valve system 168 may be operated to allow fluid to flow from the flow line 103 through the second sample point port 164 via a second inlet valve 166. When the second inlet valve 166 is opened, fluid flows through an inlet fluid line 170 to the second sample system 162. A second pressure release line 174 configured to vent a gas to atmosphere, flare, or collection, is fluidly connected to the second sample system 162. A second fluid discharge line 176 configured to discharge fluid is also fluidly connected to the second the sample system 162.

Keeping with FIG. 1C, in one or more embodiments, the first sample system 112 is fluidly connected to the flow line 103 via the first valve system 108 at a location between a choke valve 109 and a production well 102. The second sample system 162 is fluidly connected to the flow line 103 via the second valve system 168 at a location between the choke valve 109 and a gas-oil separation plant 107. While only two sample systems 112, 162 are shown in system 160, it is envisioned that any number of sample systems may be used in the systems and methods according to embodiments disclosed herein.

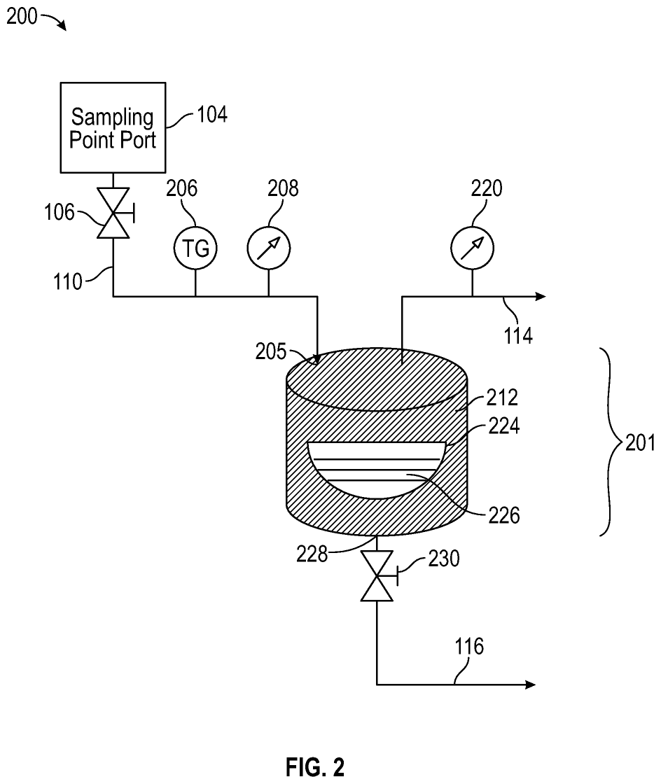

FIG. 2 shows a sample system 200 according to one or more embodiments. In one or more embodiments, the sample system 200 is the sample system 112 of FIG. 1A and FIG. 1B, or the second sample system 162 of FIG. 1C. The sample system 200 includes a sample vessel 201 fluidly connected to a sample point port 104 via an inlet valve 106, as described in FIG. 1A. An inlet port 205 on the sample vessel 201 allows a fluid to enter the sample vessel 201 through an inlet fluid line 110 when an inlet valve 106 is opened. A temperature gauge 206 and a first pressure gauge 208 are disposed on the inlet fluid line 110 at a downstream location from the inlet valve 106.

In one or more embodiments, the temperature gauge 206 is configured to measure a temperature of fluid received from the sample point port 104. Similarly, in one or more embodiments, the first pressure gauge 208 is configured to measure a pressure of fluid received from the sample point port 104. The location of the temperature gauge 206 relative to the first pressure gauge 208 is arbitrary, provided the temperature gauge 206 and the first pressure gauge 208 are located in-line with one another downstream of inlet valve 106 on the inlet fluid line 110.

The sample vessel 201 according to one or more embodiments may be constructed of any suitable material known in the art capable of containing a pressurized fluid, as described herein, and, if necessary, for handling hydrogen sulfide or other corrosive gases as may be produced from a well. For example, the sample vessel may be constructed of carbon steel, stainless steel, polymer composites, nickel alloys, Hastelloy, and the like.

Selection of the sample vessel material may depend on several factors, including the compatibility with the fluids being transported and the vessel's intended use in a specific environment. The choice of material should be made based on a thorough assessment of the fluid properties, operating conditions, and any potential corrosion or compatibility issues. Accordingly, the selected material should be both durable and safe for use in the specific application to maintain the integrity of the sample vessel and the accuracy of flow rate measurements.

The sample vessel of one or more embodiments may be constructed of carbon steel. Carbon steel is robust, durable, and suitable for a wide range of fluids. However, carbon steel may not be suitable for highly corrosive fluids. The sample vessel may also be constructed of stainless steel. Stainless steel is corrosion-resistant and may be an excellent choice for sample vessels, especially when dealing with corrosive or high-temperature fluids. Polymer composites, such as reinforced plastics or fiberglass, may also be suitable construction material for sample vessels, particularly when weight is a concern, and corrosion resistance is required. Additionally, the sample vessel may be constructed of nickel-based alloys. Nickel-based alloys may offer excellent corrosion resistance in harsh environments and may be suitable for specific applications. The sample vessel may also be constructed of Hastelloy. Hastelloy is a family of nickel-based alloys known for their excellent resistance to corrosion, making them suitable for handling highly corrosive fluids. While some options for sample vessel materials are outlined above, other suitable materials may be employed to construct the sample vessel according to one or more embodiments. The list of sample vessel materials is not intended to be taken as limiting.

The sample vessel 201 of one or more embodiments has a volume in a range of from about 3 to about 7 gallons. For example, the volume of the sample vessel may be in a range having a lower limit of from about 3, 3.5, 4, and 4.5 gallons to an upper limit of about 5, 6, and 7 gallons, where any lower limit may be paired with any upper limit.

The volume of the sample vessel may depend on several factors including the intended application, the flow rates expected from the well, and the frequency of sampling. For example, the volume of the sample vessel may depend on sampling frequency. The volume of the sample vessel according to one or more embodiments should be adequate to collect representative samples of produced fluids over a specific time interval. If the well produces fluids at a high rate, a larger sample vessel may be necessary to ensure that a sufficient volume of fluid is collected for accurate measurements. The volume of the sample vessel may also depend on desired frequency of sample collection intervals. The frequency at which samples are collected and the duration of each sampling event can influence the required sample vessel volume, for example if samples are collected infrequently, a larger vessel may be needed to capture a substantial portion of the produced fluids during each sampling event. The composition of produced fluids, including the proportions of oil, water, and gas, may also impact the sample vessel volume requirements. For example, for wells with a high water cut or gas-oil ratio, larger vessels may be necessary to accommodate varying fluid compositions. Finally, practical considerations related to the system's design, available space, and weight limitations may also influence the choice of sample vessel volume.

The sample vessel 201 of one or more embodiments should be fabricated in accordance with American Petroleum Institute (API) standards, including but not limited to all safety measures required to meet loss prevention guidelines of acid gases, such as hydrogen sulfide (H₂S) and a maximum pressure rating of 1660 psi. For example, in the oil and gas industry, there are several API guidelines and industry standards that can be referenced to ensure proper fabrication and safety of the sample vessel, including but not limited to: API Standard 520, “Sizing, Selection, and Installation of Pressure-Relieving Devices in Refineries,” API Standard 521, “Pressure-relieving and Depressuring Systems,” API Standard 526, “Flanged Steel Pressure Relief Valves,” API Standard 2000, “Venting Atmospheric and Low-pressure Storage Tanks,” API Standard 650, “Welded Tanks for Oil Storage,” API Standard 12F, “Specification for Shop Welded Tanks for Storage of Production Liquids,” API Standard 12D, “Field Welded Tanks for Storage of Production Liquids,” API Standard 620, “Design and Construction of Large, Welded, Low-pressure Storage Tanks,” API Standard 651, “Cathodic Protection of Aboveground Petroleum Storage Tanks,” API Standard 653, “Tank Inspection, Repair, Alteration, and Reconstruction,” API RP 14E, “Recommended Practice for Design and Installation of Offshore Production Platform Piping Systems,” API RP 14J, “Recommended Practice for Design and Hazards Analysis for Offshore Production Facilities, “API RP 14C, “Recommended Practice for Analysis, Design, Installation, and Testing of Basic Surface Safety Systems for Offshore Production Platforms,” API RP 67, “Recommended Practice for Oilfield Explosives Safety,” and API RP 75, “Recommended Practice for Development of a Safety and Environmental Management Program for Offshore Operations and Facilities.” These are just a selection of API guidelines and industry standards that cover various aspects of fabrication, safety, and design in the oil and gas industry and the list is in no way intended to be limiting.

The temperature gauge 206 of one or more embodiments includes any temperature gauge known in the art capable of measuring a temperature value. For example, the temperature gauge may be a contact thermometer and may be coupled with a probe or thermistor. The temperature gauge may be a digital thermometer, an analogue thermometer, a probe thermometer, a thermocouple, a thermistor, a resistance temperature detector, an infrared sensor, or the like. The measurement device used as the temperature gauge may be local or may include a transmitter to relay a signal indicative of the measurement to a remote location.

The first pressure gauge 208 of one or more embodiments includes any pressure gauge known in the art capable of measuring a pressure value. The pressure gauge may be mechanical, such as an analog type pressure gauge. Examples of analog pressure gauges include a bourdon tube and a diaphragm or bellows. The pressure gauge may also be digital and may operate using a strain gauge, piezoelectrics, and the like. The measurement device used as the pressure gauge may be local or may include a transmitter to relay a signal indicative of the measurement to a remote location.

Returning to FIG. 2, the sample vessel 201 includes a vessel body 212 and a sight glass 224 integrally formed in the vessel body 212. The sight glass 224 includes a calibrated volumetric scale 226 such that a volume of fluid and a basic sediment and water (BS&W) content contained in the sample vessel 201 may be measured. For example, undissolved solids and/or particulates may be visually observed using the sight glass upon settling to the bottom of the sample vessel. Additionally, aqueous and non-aqueous fluids may be observed upon settling in the sample vessel due to difference in the fluids' density. As the fluid sample in the sample vessel settles, denser components, such as water, tend to settle at the bottom due to their higher density, while less dense components like oil remain above. The sight glass allows operators to see the distinct layers or phases of the sample. The volume of aqueous and non-aqueous fluids may then be determined using the calibrated volumetric scale on the sight glass. The calibrated volumetric scale on the sight glass helps determine the volume of different components within the sample vessel. Operators can visually assess the volume of aqueous and non-aqueous fluids by reading the scale on the sight glass. Visual determination of BS&W using the calibrated sight glass of one or more embodiments may provide on-site assessment and monitoring of the sample composition, by using visual estimation of the BS&W content within the sample vessel.

In some embodiments, more precise BS&W measurements may be obtained using additional laboratory testing and analysis. The laboratory system may involve specialized equipment and techniques for conducting detailed analysis and separation of the components within the fluid sample, providing accurate measurements of the BS&W content. Alternatively, a conversion factor obtained from pilot testing allows for the conversion of field measurements to estimate the BS&W content under larger-scale production conditions. The conversion factor and method of determining the conversion factor is described in more detail in the following sections. This combined approach ensures precise assessment and monitoring of BS&W content in the produced fluids, contributing to effective reservoir management and operational control in the oil and gas industry.

The sample vessel 201 of one or more embodiments also includes a pressure release line 114 fluidly connected to the sample vessel 201. The pressure release line 114 contains a second pressure gauge 220 configured to measure a total vessel pressure and vent a gas from the vessel. The pressure release line 114 may also contain operable valves to control flow, release gases, etc. but may also include emergency release lines in case a high pressure slug is received. For example, a burst disc, pressure relief valve, etc. may be used for safety reasons to prevent build-up of high pressure in the sample vessel. While not explicitly shown in the drawings, one of ordinary skill in the art will recognize the inclusion of elements described herein.

In addition, the pressure release line 114 may be routed to a flare system, collection system, or to a remote/safe location such that venting of the vessel may be conducted in an appropriate manner.

In some embodiments, the vent line may be tied into the main flow line downstream of choke valves or other flow control measures in the system that may decrease a pressure of the produced fluids to below a pressure of the vent line or the pressure of the vent line when the sample system is being vented or purged. If pressures are appropriate to allow flow back into the main flow line, then the sample materials can be collected and processed along with the produced fluids.

The second pressure gauge 220 of one or more embodiments includes any pressure gauge known in the art capable of regulating a gas pressure, as described in the above sections.

The sample vessel 201 of one or more embodiments further includes an outlet port 228. The outlet port 228 fluidly connects a fluid discharge line 116 to the sample vessel 201 via an outlet valve 230. The fluid discharge line 116 may be used to remove a fluid sample from the sample vessel 201 by opening the outlet valve 230. The outlet valve 230 of one or more embodiments includes any of the valve types described above. In one or more embodiments, the fluid discharge line 116 is used to empty the sample vessel 201 to an existing slop tank if the sample system 200 is offshore or to a portable tank if the sample system 200 is onshore.

### Method for Controlling Gas-Oil Separation Plant Operations

Embodiments disclosed herein also relate to a method for controlling gas-oil separation plant operations. The method according to one or more embodiments may be applied to the systems described in the previous sections.

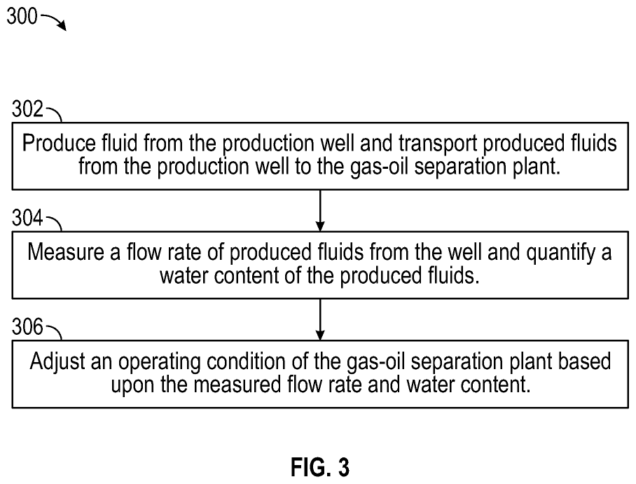

FIG. 3 shows a method 300 in accordance with one or more embodiments. The method 300 includes, in step 302, producing fluid from the production well and transporting produced fluids from the production well to the gas-oil separation plant. In some embodiments, the method for transporting produced fluids from the production well to the gas-oil separation plant further includes transporting produced fluids using a flow line, where the flow line is fluidly connected to the production well and the gas-oil separation plant.

In some embodiments, the method further includes redirecting fluid flow to a sample system, where the sample system is fluidly connected to a sample point port disposed on the flow line, using a valve system, and collecting a sample of produced fluids using the sample system, where the sample system includes a sample vessel. In some embodiments, the sample vessel includes an inlet port fluidly connected to the sample point port via an inlet valve, disposed on an inlet flow line, a temperature gauge configured to measure a temperature of fluid received from the sample point port, and a first pressure gauge configured to measure a pressure of fluid received from the sample point port. The sample vessel also includes a second pressure gauge configured to measure a total vessel pressure, fluidly connected to a pressure release line, where the pressure release line is configured to vent a gas from the sample vessel, an outlet port fluidly connected to a fluid discharge line via an outlet valve, and a sight glass having a calibrated volumetric scale, where the sight glass is integrally formed in a vessel body.

Keeping with FIG. 3, the method 300 includes, in step 304, measuring a flow rate of produced fluids from the well and quantifying a water content of the produced fluids. In some embodiments, measuring the flow rate and quantifying the water content of produced fluids further includes operating valves of a valve system to direct a total fluid flow from a flow line into a sample vessel. The method may also include recording a start time as produced fluid enters the sample vessel, collecting a volume of produced fluid within the sample vessel, operating valves of the valve system to stop fluid flow into the sample vessel and recording an end time as produced fluid stops entering the vessel. The method may also include measuring, using a calibrated volumetric scale on the sight glass located on a vessel body of the sample vessel, the volume of produced fluid collected in the sample vessel. The method may further include emptying the volume of produced fluid by opening an outlet valve disposed on an outlet port of the sample vessel.

In one or more embodiments, measuring the flow rate of produced fluids from the well and quantifying the water content of the produced fluids further includes calculating a volumetric flow rate of a volume of produced fluid at sample vessel conditions, using Equation 1:

$${Q}_{Fluid , v} = \frac{{V}_{Fluid} , v}{t} \qquad ( Equation 1 )$$

where Q_{Fluid,v} is a volumetric flow rate of the produced fluid at sample vessel conditions, V_{Fluid,v} is the collected volume of produced fluid, and t is a collection time, defined herein as a difference between a sample collection end time and a sample collection start time. Equation 1 assumes a steady-state flow condition and that the BS&W content is constant over the collection period. In one or more embodiments, measuring the flow rate of produced fluids from the well and quantifying the water content of the produced fluids further includes calculating a volumetric flow rate of oil at sample vessel conditions, using Equation 2:

$${Q}_{Oil , v} = {Q}_{Fluid , v} \times ( 1 - BSW ) \qquad ( Equation 2 )$$

where Q_{oil,v} is the volumetric flow rate of oil at sample vessel conditions, Q_{Fluid,v} is the volumetric flow rate of the fluid at sample vessel conditions, and BSW is a volume percent of basic sediment and water in the fluid. Equation 2 assumes a steady-state flow condition and that the BS&W content is constant over the collection period.

In one or more embodiments, measuring the flow rate of produced fluids from the well and quantifying the water content of the produced fluids further includes calculating a volumetric flow rate of water at sample vessel conditions, using Equation 3:

$${Q}_{Water , v} = \frac{{Q}_{Fluid , v}}{BSW} \qquad ( Equation 3 )$$

where Q_{water,v} is a volumetric flow rate of water at sample vessel conditions, Q_{Fluid,v} is the volumetric flow rate of the produced fluid at sample vessel conditions, and BSW is the volume percent of basic sediment and water (BS&W) in the produced fluid. Equation 3 assumes a steady-state flow condition and that the BS&W content is constant over the collection period.

In some embodiments, the method for measuring the flow rate of produced fluids from the well and quantifying the water content of the produced fluids further includes determining, using a laboratory system or using production data, a gas-oil ratio (GOR) and calculating a volumetric flow rate of gas at as-produced conditions using Equation 4:

$${Q}_{GAS} = {R}_{oil} \times GOR \qquad ( Equation 4 )$$

where Q_{GAS} is the volumetric flow rate of gas at as produced conditions, Rₒᵢₗ is a production rate of oil, and GOR is a gas-oil ratio. The production rate of oil may be obtained from production data.

Calculation of the volumetric flow rate of gas using Equation 4 may provide a comprehensive analysis of produced fluids from the well, which includes not only the water content (BS&W) and the oil flow rate but also the gas component. By calculating the volumetric flow rate of gas, a more complete understanding of the entire fluid composition may be obtained. Furthermore, understanding the gas-oil ratio (GOR) is crucial for reservoir management and production optimization in the oil and gas industry. The GOR represents the ratio of gas production to oil production and is an important parameter for evaluating the performance of a well and the reservoir itself. The GOR calculation also provides valuable information for operational control necessary to make decisions related to well production strategies, adjusting choke settings, and optimizing the separation and handling of gas and oil streams in the field. The calculated GOR can be compared to GOR values obtained from production data, which serves as a validation step to ensure that the measurements and calculations made using the sample system align with the actual production data, providing confidence in the accuracy of the system. Additionally, in oilfield operations, a complete understanding of the fluid composition, including gas, oil, and water phases, is essential for reservoir characterization. Reservoir characterization information supports reservoir modeling and simulation, which is critical for long-term reservoir development and management. In summary, the calculation of the volumetric flow rate of gas using Equation 4 contributes to a more comprehensive and accurate characterization of the produced fluids from the well by aiding in reservoir management, operational control, and data validation, ensuring that the invention provides a holistic approach to fluid analysis in the oil and gas industry.

### As-Produced Condition Corrected Flow Rates

One or more embodiments relates to a method for correcting a volumetric flow rate of fluid at sample vessel conditions to a volumetric flow rate at as-produced conditions. In order to determine a volumetric flow rate at as-produced conditions, sample vessel readings from small-scale conditions (e.g., ¼″ OD pipe) may be converted to larger-scale production and/or stock tank conditions (e.g., 3″ production). Calculation of a conversion factor according to one or more embodiments may allow accurately scale-up of measurements obtained in the small pipe to represent conditions in a larger production system.

According to embodiments disclosed herein, “sample vessel conditions” refer to volumes and flow rates collected through the inlet fluid flow line to the sample system having a specific internal pipe diameter (ID) and “as-produced conditions” refer to volumes and flow rates collected through the production flow line having a specific internal pipe diameter (ID), as shown FIGS. 1A-1C.

In some embodiments, the method for measuring the flow rate of produced fluids from the well and quantifying the water content of the produced fluids further includes calculating, using a conversion factor, a volumetric flow rate of the volume of produced fluid at as-produced conditions using Equation 5:

$${Q}_{Fluid} = {Q}_{Fluid , v} \times C \qquad ( Equation 5 )$$

where Q_{Fluid} is a volumetric flow rate of the volume of produced fluid at as-produced conditions, Q_{Fluid,v} is the volumetric flow rate of the fluid at sample vessel conditions, and C is a conversion factor.

In one or more embodiments, the conversion factor may be determined through pilot testing at different wells. During pilot tests, measurements are taken simultaneously in both the small-scale (e.g., ¼″ OD pipe) and large-scale (e.g., 3″ production) conditions. For the pilot testing, measurements would be collected for parameters such as flow rates, pressures, temperatures, and other relevant variables in both the small-scale and large-scale conditions.

After conducting multiple pilot tests and collecting sufficient data, a correlation would be developed. This correlation would relate the measurements obtained in the small-scale conditions to an equivalent value in the larger-scale conditions. The conversion factor is essentially the mathematical relationship that allows you to convert from one scale to the other. The conversion factor, C, is a ratio of the volumetric flow rate of the volume of produced fluid at as-produced conditions, Q_{Fluid}, to the volumetric flow rate of the fluid at sample vessel conditions, Q_{Fluid,v}, as shown in Equation 5.

### Temperature and Pressure Corrected Flow Rates

One or more embodiments relates to a method for correcting a volumetric flow rate of fluid at as-produced conditions to a corrected volumetric flow rate at standard temperature and pressure conditions. Because temperature and pressure changes affect the density of a fluid, temperature and pressure changes can affect the accuracy of flow rate calculations. To account for this, the volume of liquid collected may be corrected to a standard temperature and pressure (STP) condition. According to embodiments disclosed herein, the Real Gas Law or other specialized equations of state may be used to determine a corrected volume by accounting for the behavior of real, non-ideal gases and liquids under various temperature and pressure conditions. The corrected volume can then be used in the calculation of the volumetric flow rate.

According to embodiments disclosed herein, “as-produced temperature” conditions and “as-produced pressure” conditions refer to temperatures and pressures of a produced fluid as it enters the sample vessel. The as-produced temperature and as-produced pressure may be measured using a temperature gauge and a first pressure gauge, as described in more detail in the discussion of FIGS. 1A-1C, above.

According to embodiments disclosed herein, “standard temperature” conditions refer to a temperature of 60° F. (519.67° R, 15.55° C.) and “standard pressure” conditions refer to a pressure of 14.7 psi (1 atm).

In some embodiments, the method for controlling gas-oil separation plant operations further includes calculating a temperature-corrected volume of produced fluid at a standard temperature, using Equation 6:

$${V}_{STD , T} = {{V}_{Fluid , v} ( \frac{{T}_{prod}}{{T}_{STD}} )}^{( 1 - γ )} \qquad ( Equation 6 )$$

where V_{STD,T} is the temperature-corrected volume of the produced fluid at standard temperature, V_{Fluid} is the collected volume of the produced fluid, T_{prod} is a temperature of the produced fluid as collected, T_{STD} is the standard temperature, and γ is a ratio of specific heats of gases. Application of Equation 6 assumes that the pressure remains constant during the collection period for which a volume is collected in the sample vessel.

According to one or more embodiments, γ is a ratio of specific heats (i.e., heat capacity) of a gas. γ is a thermodynamic property of gases that characterizes how the gas temperature changes when it is subjected to changes in pressure and volume while keeping its internal energy constant. The ratio of specific heats (γ) is defined by Equation (7):

$$γ = \frac{{C}_{p}}{{C}_{v}} \qquad ( Equation 7 )$$

where Cₚ is a specific heat capacity at constant pressure (heat required to increase the temperature of the gas at constant pressure) of a gas, and Cᵥ is a specific heat capacity at constant volume (heat required to increase the temperature of the gas at constant volume) of the gas.

In thermodynamics, most ideal gases have a γ of approximately 1.4. This value is a commonly used approximation for monoatomic gases, such as helium (He) and diatomic gases, such as nitrogen (N2) and oxygen (O2), under standard conditions. However, the value of γ can vary slightly for different gases and under extreme conditions. In the context of the provided equation (Equation 5), γ is used to account for the temperature correction of the volume of produced fluid to a standard temperature (T_{STD}). This correction is necessary to ensure consistency and accuracy in flow rate calculations, particularly when dealing with gases that may undergo significant temperature changes during production and processing.

In some embodiments, the method for controlling gas-oil separation plant operations further includes calculating a pressure-corrected volume of produced fluid at a standard pressure, using Equation 8:

$${V}_{STD} = {V}_{STD , T} ( \frac{{P}_{prod}}{{P}_{STD}} ) \qquad ( Equation 8 )$$

where V_{STD} is the pressure-corrected and temperature-corrected volume of the produced fluid at standard pressure, V_{STD,T} is the temperature-corrected volume of the produced fluid at standard temperature, P_{prod} is a pressure of the produced fluid as collected, and P_{STD} is a standard pressure.

In one or more embodiments, Equation 8 is a simplified form of Boyle's Law, which describes the inverse relationship between the pressure and volume of a gas when the temperature is held constant. Boyle's Law is appropriate for ideal gases, and this equation is often used when dealing with the pressure correction of liquids in a closed system. Application of Boyle's law assumes that the temperature remains constant during the collection period for which a volume is collected in the sample vessel.

Assumptions associated with the abovementioned equations, for example ideal fluid behavior, constant pressure conditions and constant pressure conditions may provide reasonable approximations of fluid behavior according to one or more embodiments when used in many practical situations, especially when dealing with ideal gases or when small changes in temperature or pressure occur within the system.

In some embodiments, correcting the volume of liquids in produced fluids to standard temperature and pressure (STP) conditions, may include using the real gas law or using one or more thermodynamic relationships, for example, equations of state. The Real Gas Law includes corrections for non-ideal behavior of fluids and can provide a more accurate representation when dealing with volume changes of liquids due to changes in temperature and pressure, by considering factors such as molecular interactions and compressibility. Additionally, instead of approximating γ as having a value of 1.4, γ may be more precisely calculated using thermodynamic tables or databases for various gases under different conditions when dealing with non-ideal gases. In some embodiments, correcting the volume of liquids in produced fluids to standard temperature and pressure (STP) conditions using the real gas law or using one or more thermodynamic relationships may involve specialized equations or software tools tailored to the behavior of real fluids under varying conditions. The exact equations and thermodynamic relationships used to correct the volume of liquids in produced fluids according to one or more embodiments may vary depending on the fluid composition and the specific industry standards followed by the oil and gas company.

In some embodiments, upon calculating a volume of the produced fluid at standard conditions, the method may further include calculating a volumetric flow rate of the volume of produced fluid at standard conditions, using Equation 9:

$${Q}_{STD} = \frac{{V}_{STD}}{t} \qquad ( Equation 9 )$$

where Q_{STD} is the volumetric flow rate of the fluid at standard conditions, V_{STD} is the volume of the produced fluid at standard conditions, and t is the collection time.

In some embodiments, the method further includes calculating a volumetric flow rate of oil at standard conditions, using Equation 10:

$${Q}_{oil , STD} = \frac{{Q}_{STD}}{( 1 - BSW )} \qquad ( Equation 10 )$$

where, Q_{Oil,STD} is the volumetric flow rate of oil at standard conditions, Q_{Fluid} is the volumetric flow rate of produced fluid at standard conditions, and BSW is the volume percent of basic sediment and water in the fluid.

In some embodiments, the method further includes calculating a volumetric flow rate of water at standard conditions, using Equation 11:

$${Q}_{Water , STD} = {Q}_{STD} \times BSW \qquad ( Equation 11 )$$

where, Q_{Water,STD} is the volumetric flow rate of water at standard conditions, Q_{STD} is the volumetric flow rate of produced fluid at standard conditions, and BSW is the volume percent of basic sediment and water in the fluid.

The volumetric flow rate of water and the volumetric flow rate of oil calculated using the temperature and pressure corrections according to one or more embodiments are thus accurate even if the temperature and pressure of produced fluid vary.

In some embodiments, the method further includes redirecting fluid flow to a second sample system, wherein the second sample system is fluidly connected to a second sample point port disposed on the flow line, using a second valve system, and collecting a second sample of produced fluids using the second sample system, where the second sample system includes a second sample vessel.

In some embodiments, upon redirecting fluid flow to a second sample system and collecting a second sample of produced fluids using the second sample system, the method further includes calculating an average volumetric flow rate of a produced fluid at as-produced conditions over a length of a flow line, using Equation 12:

$${Q}_{Average} = ( \frac{{V}_{Fluid , 1}}{{t}_{1}} + \frac{{V}_{Fluid , 2}}{{t}_{2}} ) / 2 \qquad ( Equation 12 )$$

where Q_{Average} is the average volumetric flow rate of produced fluid at as-produced conditions, V_{Fluid,1} is a volume of produced fluid at as-produced conditions collected using the sample system, t₁ is a first collection time, V_{Fluid,2} is a volume of produced fluid at as-produced conditions collected using the second sample system, and t₂ is a second collection time. The length of the flow line is defined as a distance between the sample point port and the second sample point port.

In one or more embodiments, the use of multiple sample systems disposed alone multiple points along the flow line is used to improve flow rate accuracy. For example, collecting multiple production fluid samples along the flow line may provide a more accurate representation of the flow rate of production fluid within the flow line than only collecting a single sample. An average flow rate across the flow line may therefore be calculated, allowing for an overall improved measurement which may be less susceptible to measurement errors or fluctuations in the flow and may help to account for any variations in the flow rate that may occur along the length of the pipeline.

Returning to FIG. 3, the method 300 includes, in step 306, adjusting an operating condition of the gas-oil separation plant based upon the measured flow rate and water content. In some embodiments, adjusting an operating condition of the gas-oil separation plant includes adjusting one or more of a flow rate, a chemical injection rate, a separator vessel pressure level, a gas compression ratio, a settling time, a pH level, a concentration of emulsion breaker, a heat exchanger temperature, a level control, a flow path routing, a sampling frequency, an equipment maintenance schedule, a gas composition, a water disposal plan, and a water treatment plan.

In one or more embodiments, adjusting a flow rate includes using a flow control valves in the system to regulate the flow of fluids and optimize separation efficiency. Adjusting a chemical injection rates may include adjusting the rates at which chemicals are injected into the process. Adjusting a separator vessel pressure level may include monitoring and controlling pressure levels within a separator vessels to ensure efficient phase separation and avoid issues like foaming or carryover. Adjusting a gas compression ratio may include adjusting a compression ratio and condition to accommodate changes in gas flow rates. Adjusting a settling time may include adjusting, based on operating conditions, a duration for which fluids are allowed to settle in the separator vessel can impact separation efficiency. Adjusting a pH level may include, in cases where pH adjustment is relevant to the separation process, adjusting and/or monitoring the pH level of fluids used in the separation process. Adjusting a concentration of emulsion breaker may include, if emulsion breakers or demulsifiers are used, adjusting the dosage and injection points of the emulsion breakers to enhance phase separation. Adjusting a heat exchanger temperature may include controlling a heat exchanger temperature to optimize the separation of different phases, especially in cold or high-temperature environments. Adjusting a level control may include maintaining proper fluid levels in the separator vessels to ensure effective separation and prevent carryover of undesired phases. Adjusting a flow path routing may include determining the routing of produced fluids through different processing units or vessels to achieve the desired separation outcomes. Adjusting a sampling frequency may include adjusting how often samples are collected and analyzed to monitor fluid properties and adjust operating conditions accordingly. Adjusting an equipment maintenance schedules may include establishing regular maintenance intervals for key equipment components to ensure they operate optimally. Adjusting a gas composition may include, gas composition adjustments based on variations in the composition of the produced gas. Adjusting a water disposal plan may include adjusting methods and rates at which water is disposed. Adjusting a water treatment plan may include adjusting methods and rates at which water is treated. In one or more embodiments, conditions can vary depending on the specific design and requirements of the gas-oil separation plant and the nature of the produced fluids. Adjusting conditions, for example those conditions outlined above, according to one or more embodiments may ensure that the system operates efficiently and meets the desired separation goals.

Additional considerations for conducting methods using the systems according to one or more embodiments disclosed herein include sampling frequency and maintenance. As an illustrative example, the frequency at which samples are collected must be determined based on the specifics of the pipeline and the flow conditions. If the samples are collected too infrequently, fluctuations in the flow rate may not be accurately represented. Collecting samples too frequently may become time-consuming and lead to increased cost of operations. Actual sampling frequency may vary depending on production well conditions, such as pipeline length and complexity for example. Maintenance of equipment used for sample collection and measurement must be properly maintained to ensure accurate and reliable results. This includes regular calibration, cleaning, and replacement of parts as needed.

In some embodiments, the collected data from sample collection points may be analyzed using advanced data analysis techniques including one or more of the following to further improve the accuracy of the flow rate measurement: data smoothing, statistical analysis, quality control, calibration and validation, historical data comparison, and machine learning and AI methods.

Analyzing collected data using data smoothing techniques according to one or more embodiments may include removing noise or irregularities from the collected data to obtain a more stable and accurate representation of the flow rate. Various algorithms, such as moving averages or exponential smoothing, can be applied to the data to reduce fluctuations caused by measurement errors or transient conditions. Analyzing collected data using statistical analysis according to one or more embodiments may include analyzing the collected data for patterns, trends, and anomalies to help identify systematic errors or variations in flow rates that may not be immediately apparent from raw data. Statistical techniques like regression analysis may also be employed to establish relationships between different variables that affect flow rates. Analyzing collected data using quality control techniques according to one or more embodiments may include applying rigorous quality control procedures to ensure the integrity of the collected data. Applying rigorous quality control procedures may involve identifying and filtering out outliers or data points that do not conform to expected patterns. In addition, data quality metrics and checks can be established to flag potential issues in real-time. Analyzing collected data using calibration and validation techniques according to one or more embodiments may include regular calibration of measurement equipment and validation of data against known standards or reference measurements to ensure that the measurement system remains accurate and reliable over time. Analyzing collected data using historical data comparison according to one or more embodiments may include comparing current flow rate measurements with historical data to provide insights into long-term trends and changes in the flow behavior and help operators make informed decisions and anticipate future operational challenges. Analyzing collected data using machine learning and AI methods according to one or more embodiments may include applying advanced machine learning and artificial intelligence (AI) algorithms to the data for predictive modeling and anomaly detection. Machine learning and AI techniques can learn from historical data to make real-time predictions and detect deviations from expected flow rate patterns.

Including these data analysis techniques in the documentation highlights the potential for improving the accuracy and reliability of flow rate measurements, especially when dealing with complex and dynamic fluid flow conditions. It underscores the importance of not only collecting data but also processing and interpreting it effectively to support decision-making, optimize production, and ensure safety in industries like oil production and transportation.

Systems and methods according to one or more embodiments disclosed herein may include the following advantages.

The methods and systems described according to embodiments herein may resolve and may be used to support data in the event of any doubt of rate tests arising from multi-phase flowmeter (MPFM) discrepancy or MPFM in need of calibration. Data acquired according to methods and systems disclosed herein may be more accurate data since it is gauged data not measured or calculated based on human algorithm or correlation.

The method of measuring flow rates from sample collection points according to one or more embodiments may be used to accurately measure the flow rate of a liquid in a pipeline. Measuring fluid flow rates is a critical aspect of industries such as oil production and transportation, where accurate flow rate measurement may be used in order to optimize production and ensure safe and efficient transport of produced fluids. Accurate flow rate measurement is important because it allows engineers and operators to monitor the flow of liquids in real-time and make informed decisions about production and transportation. Inaccurate flow rate measurements can result in reduced efficiency, increased costs, and potentially hazardous situations.

By using the method according to one or more embodiments, engineers and operators can determine the flow rate with a high degree of accuracy, even in pipelines that are long and complex. The method may help to minimize measurement errors and provides a more accurate representation of the flow rate, allowing for better decision-making and improved operations. By collecting multiple samples and averaging the results, the measurement of flow rates from sample collection points according to one or more embodiments may be less susceptible to measurement errors or fluctuations in the flow, leading to a more accurate representation of the flow rate.

By using multiple sample collection points in the systems and methods of one or more embodiments disclosed herein, any variations in the flow rate that may occur along the length of the pipeline may be accounted for. Use of multiple sample points therefore provides a more comprehensive view of the flow rate than would be possible with a single measurement. In addition, multiple sample collection points may provide a better representation of the flow conditions in a pipeline by collecting multiple samples over time, which is particularly important in pipelines that are long and complex, where there may be significant variations in the flow rate.

Systems and methods of one or more embodiments disclosed herein may also improve the safety of personnel involved in the measurement process. For example, the use of sample collection points and accurate flow meters reduces the need for direct access to the pipeline, which may be hazardous in certain situations.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A system for measuring a fluid flow rate from a well, including a water flow rate, the system comprising:
a production well;
a flow line for transporting produced fluids from the production well to a gas-oil separation plant;
a sample system comprising a sample vessel fluidly connected to a sample point port disposed on the flow line;
wherein the sample vessel comprises,
an inlet port fluidly connected to the sample point port via an inlet valve, disposed on an inlet flow line;
a temperature gauge configured to measure a temperature of fluid received from the sample point port;
a first pressure gauge configured to measure a pressure of fluid received from the sample point port;
a second pressure gauge configured to measure a total vessel pressure, fluidly connected to a pressure release line, wherein the pressure release line is configured to vent a gas from the sample vessel;
an outlet port fluidly connected to a fluid discharge line via an outlet valve;
a sight glass comprising a calibrated volumetric scale, wherein the sight glass is integrally formed in a vessel body;
a valve system for redirecting fluid flow from the flow line to the sample vessel via the sample point port.

2. The system of claim 1, wherein the sample system further comprises a second sample vessel disposed upstream or downstream of the sample vessel along the flow line, wherein the second sample vessel is fluidly connected to a second sample point port disposed on the flow line.

3. The system of claim 2, wherein the sample point port is upstream of a choke valve of the production well and the second sample point port is downstream of the choke valve.

4. The system of claim 1, wherein the sample point port is upstream of a choke valve of the production well.

5. The system of claim 1, wherein the sample vessel has a maximum pressure rating of 1660 psi.

6. The system of claim 1, wherein a volume of the sample vessel is in a range of from 3 gallons to 7 gallons.

7. The system of claim 1, wherein the sample vessel comprises carbon steel, stainless steel, polymer composites, nickel alloys, or Hastelloy.

8. A method for controlling gas-oil separation plant operations using the system of claim 1, comprising:
producing fluid from the production well and transporting produced fluid from the production well to the gas-oil separation plant;
measuring a flow rate of produced fluid from the well and quantifying a water content of the produced fluid; and
adjusting an operating condition of the gas-oil separation plant based upon the measured flow rate and water content.

9. The method of claim 8, wherein the produced fluid comprises a mixture of at least oil and water.

10. The method of claim 8, wherein measuring the flow rate of produced fluid from the well and quantifying the water content of the produced fluid comprises:
operating valves of the valve system to direct a total fluid flow from the flow line into the sample vessel;
recording a start time;
collecting a volume of produced fluid within the sample vessel;
operating valves of the valve system to stop fluid flow into the sample vessel;
recording an end time;
measuring, using the calibrated volumetric scale on the sight glass, the volume of produced fluid collected in the sample vessel; and
emptying the volume of produced fluid by opening an outlet valve disposed on an outlet port of the sample vessel.

11. The method of claim 10, wherein measuring the flow rate of produced fluid from the well and quantifying the water content of the produced fluid further comprises:
determining, using the calibrated volumetric scale on the sight glass, a volume percent of basic sediment and water;
calculating a volumetric flow rate of the volume of produced fluid at sample vessel conditions;
calculating a volumetric flow rate of oil at sample vessel conditions; and
calculating a volumetric flow rate of water at sample vessel conditions.

12. The method of claim 11, further comprising:
calculating, using a conversion factor, a volumetric flow rate of the volume of produced fluid at as-produced conditions.

13. The method of claim 12, further comprising:
calculating a temperature-corrected volume of produced fluid at a standard temperature.

14. The method of claim 13, further comprising:
calculating, using the temperature-corrected volume of produced fluid at standard temperature, a pressure-corrected and temperature-corrected volume of produced fluid at a standard pressure.

15. The method of claim 14, further comprising:
calculating, based on the volume of produced fluid at standard conditions, a flow rate of produced fluid at standard conditions;
calculating a volumetric flow rate of oil at standard conditions; and
calculating a volumetric flow rate of water at standard conditions.

16. The method of claim 8, wherein transporting produced fluid from the production well to the gas-oil separation plant further comprises transporting produced fluid using a flow line, wherein the flow line is fluidly connected to the production well and the gas-oil separation plant.

17. The method of claim 16, further comprising:
redirecting fluid flow to a sample system, wherein the sample system is fluidly connected to a sample point port disposed on the flow line, using a valve system; and
collecting a sample of produced fluid using the sample system, comprising a sample vessel, wherein the sample vessel comprises;
an inlet port fluidly connected to the sample point port via an inlet valve, disposed on an inlet flow line;
a temperature gauge configured to measure a temperature of fluid received from the sample point port;
a first pressure gauge configured to measure a pressure of fluid received from the sample point port;
a second pressure gauge configured to measure a total vessel pressure, fluidly connected to a pressure release line, wherein the pressure release line is configured to vent a gas from the sample vessel;
an outlet port fluidly connected to a fluid discharge line via an outlet valve; and
a sight glass comprising a calibrated volumetric scale, wherein the sight glass is integrally formed in a vessel body.

18. The method of claim 17, further comprising:
redirecting fluid flow to a second sample system, wherein the second sample system is fluidly connected to a second sample point port disposed on the flow line, using a second valve system; and
collecting a second sample of produced fluid using the second sample system, comprising a second sample vessel.

19. The method of claim 18, further comprising:
calculating an average volumetric flow rate of a produced fluid at as-produced conditions over a length of the flow line, wherein the length of the flow line comprises a distance between the sample point port and the second sample point port.

20. The method of claim 8, wherein adjusting an operating condition of the gas-oil separation plant comprises adjusting one or more of a flow rate, a chemical injection rate, a separator vessel pressure level, a gas compression ratio, a settling time, a pH level, a concentration of emulsion breaker, a heat exchanger temperature, a level control, a flow path routing, a sampling frequency, an equipment maintenance schedule, a gas composition, a water disposal plan, and a water treatment plan.
